(19) United States
(12) Patent Application Publication
(10) Pub. No.: US 2013/0331458 A1
(43) Pub. Date: Dec. 12, 2013

(54) KIT OF OPHTHALMIC COMPOSITION

(71) Applicant: ROHTO PHARMACEUTICAL CO., LTD., (US)

(72) Inventors: Takayuki MIYANO, Osaka (JP); Takahiro KUROSE, Osaka (JP)

(73) Assignee: ROHTO PHARMACEUTICAL CO., LTD., Osaka (JP)

(21) Appl. No.: 13/775,547

(22) Filed: Feb. 25, 2013

Related U.S. Application Data
(60) Provisional application No. 61/638,734, filed on Apr. 26, 2012.

(30) Foreign Application Priority Data
Feb. 27, 2012 (JP) 2012-040548

(51) Int. Cl. A61K 31/121

(52) U.S. Cl. CPC A61K 31/121
USPC 514/675

(57) ABSTRACT

An ophthalmic composition kit comprising an ophthalmic container holding an ophthalmic composition comprising geranylgeranylacetone, the ophthalmic container having a surface in contact with the ophthalmic composition, the surface being at least partially or wholly made of a container material selected from the group consisting of a polyolefin, an acrylic acid resin, a terephthalic acid ester, a polycarbonate, a polymethylterpene, a fluorine resin and a glass,

the ophthalmic composition kit having an advantage that the loss of the geranylgeranylacetone content or decrease in the geranylgeranylacetone concentration in the ophthalmic composition is very little.

### TECHNICAL FIELD

The present invention relates to an ophthalmic composition kit comprising an ophthalmic container holding an ophthalmic composition comprising geranylgeranylacetone.

### BACKGROUND ART

Teprenone (Eisai Co., Ltd.) is a mixture of (5E,9E,13E)-geranylgeranylacetone (hereinafter sometimes referred to as “all-trans form”) and (5Z,9E,13E)-geranylgeranylacetone (hereinafter sometimes referred to as “5Z-mono-cis form”) at a weight ratio of 3:2. Teprenone is widely used as an oral therapeutic agent for gastric ulcer.

The use of teprenone in the ophthalmic field has been suggested. For example, Patent Literature 1 teaches the use of teprenone as an active ingredient of a prophylactic or therapeutic agent for dry eye, eye strain, or eye dryness.

Patent Literature 2 discloses a clear eye drop consisting of teprenone, a phospholipid, a synthetic surfactant, and water.

However, there has been a problem that since geranylgeranylacetone tends to be adsorbed to the inner wall of a container, the geranylgeranylacetone content tends to be reduced during storage.

### CITATION LIST

### Patent Literature

Patent Literature 1: JP-8-133967 A
Patent Literature 2: JP-2000-319170 A

### SUMMARY OF INVENTION

### Technical Problem

An object of the present invention is to provide an ophthalmic composition kit comprising an ophthalmic container holding an ophthalmic composition comprising geranylgeranylacetone, the kit being characterized by that the loss of the geranylgeranylacetone content of the ophthalmic composition is reduced.

### Solution to Problem

The inventors conducted extensive research in order to solve the above problem and found that the loss of the geranylgeranylacetone (hereinafter sometimes referred to as “GGA”) content of an ophthalmic composition is remarkably reduced when the ophthalmic composition comprising GGA is held by an ophthalmic container whose part in contact with the ophthalmic composition is made of at least one material selected from the group consisting of a polyolefin, an acrylic acid resin, a terephthalic acid ester, a 2,6-naphthalene dicarboxylic acid ester, a polycarbonate, a polymethylterpene, a fluorine resin, a polyvinyl chloride, a polyamide, an ABS resin, an AS resin, a polyacetal, a modified polyphenylene ether, a polyarylate, a polysulfone, a polyimide, a cellulose acetate, a hydrocarbon optionally substituted with a halogen atom, an aluminum and a glass.

The present invention has been completed based on the above finding and provides an ophthalmic composition kit etc., as described below.

(1) An ophthalmic composition kit comprising an ophthalmic container holding an ophthalmic composition comprising geranylgeranylacetone,

the ophthalmic container having a surface in contact with the ophthalmic composition, the surface being at least partially or wholly made of at least one container material selected from the group consisting of a polyolefin, an acrylic acid resin, a terephthalic acid ester, a polycarbonate, a polymethylterpene, a fluorine resin and a glass.

(2) The ophthalmic composition kit according to the above (1), wherein the container material is at least one selected from the group consisting of a polyethylene, a polypropylene, a polymethyl methacrylate, a polyethylene terephthalate, a polycarbonate, a polymethylterpene, a polytetrafluoroethylene and a glass.

(3) The ophthalmic composition kit according to the above (1) or (2), wherein the geranylgeranylacetone content of the ophthalmic composition is 0.00001 to 10% by weight relative to the total amount of the composition.

(4) The ophthalmic composition kit according to any of the above (1) to (3), wherein the pH of the ophthalmic composition is from 6 to 8.

(5) The ophthalmic composition kit according to any of the above (1) to (4), wherein the ophthalmic composition further comprises a phosphate buffering agent.

(6) The ophthalmic composition kit according to any of the above (1) to (5), wherein the ophthalmic composition further comprises a fat-soluble antioxidant.

(7) The ophthalmic composition kit according to any of the above (1) to (6), wherein the ophthalmic composition is in the form of a liquid, a fluid or a semi-solid.

(8) The ophthalmic composition kit according to any of the above (1) to (6), wherein the ophthalmic composition is an eye drop and the ophthalmic container is an eye drop container.

(9) A method for reducing the loss of the geranylgeranylacetone content of an ophthalmic composition, the method comprising the step of employing, as an ophthalmic container for holding the ophthalmic composition comprising geranylgeranylacetone, a container whose surface in contact with the ophthalmic composition is at least partially or wholly made of at least one container material selected from the group consisting of a polyolefin, an acrylic acid resin, a terephthalic acid ester, a polycarbonate, a polymethylterpene, a fluorine resin and a glass,

thereby reducing the loss of the geranylgeranylacetone content of the ophthalmic composition.

(10) A method for reducing adsorption of geranylgeranylacetone to a wall of an ophthalmic container, the method comprising the step of employing, as an ophthalmic container for holding an ophthalmic composition comprising geranylgeranylacetone, a container whose surface in contact with the ophthalmic composition is at least partially or wholly made of at least one container material selected from the group consisting of a polyolefin, an acrylic acid resin, a terephthalic acid ester, a polycarbonate, a polymethylterpene, a fluorine resin and a glass,

thereby reducing adsorption of geranylgeranylacetone to the wall of the ophthalmic container.

### Advantageous Effects of Invention

In the ophthalmic composition kit of the present invention, since the ophthalmic composition is held by an ophthalmic container whose surface in contact with the ophthalmic composition is partially or wholly made of some kind of material, the loss of the GGA content or decrease in the GGA concentration in the ophthalmic composition is very little. The loss of the GGA content varies depending on the container material and hence it is considered that the use of a container material of some kind remarkably reduces adsorption of GGA to a container wall.

### BRIEF DESCRIPTION OF DRAWINGS

FIG. 1 shows the appearance of the eye drop containers used in Examination for residual ratio of GGA No. 2.

### DESCRIPTION OF EMBODIMENTS

The present invention will be described in detail below.

The ophthalmic composition kit of the present invention is an ophthalmic composition kit comprising an ophthalmic container holding an ophthalmic composition comprising geranylgeranylacetone, the ophthalmic container having a surface in contact with the ophthalmic composition, the surface being at least partially or wholly made of at least one container material selected from the group consisting of a polyolefin, an acrylic acid resin, a terephthalic acid ester, a 2,6-naphthalene dicarboxylic acid ester, a polycarbonate, a polymethylterpene, a fluorine resin, a polyvinyl chloride, a polyamide, an ABS resin, an AS resin, a polyacetal, a modified polyphenylene ether, a polyarylate, a polysulfone, a polyimide, a cellulose acetate, a hydrocarbon optionally substituted with a halogen atom, an aluminum and a glass.

### Geranylgeranylacetone

### (1) Types of Geometric Isomers

GGA has eight geometric isomers. Specifically, the eight geometric isomers are:
(5E,9E,13E)-6,10,14,18-tetramethyl-5,9,13,17-nonadecatetraen-2-one (5E,9E,13E GGA) (all-trans form),
(5Z,9E,13E)-6,10,14,18-tetramethyl-5,9,13,17-nonadecatetraen-2-one (5Z,9E,13E GGA) (5Z-mono-cis form),
(5Z,9Z,13E)-6,10,14,18-tetramethyl-5,9,13,17-nonadecatetraen-2-one (5Z,9Z,13E GGA) (13E-mono-trans form),
(5Z,9Z,13Z)-6,10,14,18-tetramethyl-5,9,13,17-nonadecatetraen-2-one (5Z,9Z,13Z GGA) (all-cis form),
(5E,9Z,13E)-6,10,14,18-tetramethyl-5,9,13,17-nonadecatetraen-2-one (5E,9Z,13E GGA) (9Z-mono-cis form),
(5E,9Z,13Z)-6,10,14,18-tetramethyl-5,9,13,17-nonadecatetraen-2-one (5E,9Z,13Z GGA) (5E-mono-trans form),
(5E,9E,13Z)-6,10,14,18-tetramethyl-5,9,13,17-nonadecatetraen-2-one (5E,9E,13Z GGA) (13Z-mono-cis form),
and
(5Z,9E,13Z)-6,10,14,18-tetramethyl-5,9,13,17-nonadecatetraen-2-one (5Z,9E,13Z GGA) (9E-mono-trans form).

These GGAs can be used alone or in any combination of two or more thereof according to the present invention. In cases where two or more of the GGAs are combined, the mixing ratio is not particularly limited.

Among the above GGAs, preferred are the all-trans form, the mono-cis forms (especially the 5Z-mono-cis form) and a mixture of the all-trans form and one of the mono-cis forms.

In cases where the GGA of the present invention is a mixture of the all-trans form and one of the mono-cis forms (especially the 5Z-mono-cis form), the all-trans form content of the mixture is preferably 80% by weight or more, more preferably 82% by weight or more, further more preferably 84% by weight or more, further more preferably 86% by weight or more, further more preferably 88% by weight or more, further more preferably 90% by weight or more, further more preferably 92% by weight or more, further more preferably 94% by weight or more, further more preferably 96% by weight, further more preferably 98% by weight or more. Especially preferably, the GGA consists of the all-trans form. When the all-trans form is in the above ranges, white turbidity at low temperature is reduced.

Also preferred GGA is a mixture of the all-trans form and one of the mono-cis forms (especially the 5Z-mono-cis form) with a very high mono-cis form (especially the 5Z-mono-cis form) ratio.

### (2) All-Trans Form and 5Z-Mono-Cis Form

(5E,9E,13E)-geranylgeranylacetone (the all-trans form) is a compound represented by the following structural formula:

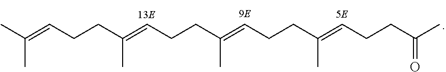 [SMILES: CC(=O)CC/C=C(\C)CC/C=C(\C)CC/C=C(\C)CCC=C(C)C]

The all-trans form can be purchased from, for example, Rionlon Development Co., Ltd.

The all-trans form can also be obtained through separating the all-trans form and the 5Z-mono-cis form of a marketed teprenone (Eisai Co., Ltd., Wako Pure Chemical Industries, Ltd., Yoshindo Inc., etc.) by, for example, silica gel chromatography using a mobile phase of n-hexane/ethyl acetate (9:1). The separation of the all-trans form and the 5Z-mono-cis form of a marketed teprenone can also be commissioned to, for example, KNC Laboratories Co., Ltd.

(5Z,9E,13E)-geranylgeranylacetone (the 5Z-mono-cis form) can also be obtained by the separation from a marketed teprenone. The 5Z-mono-cis form is a compound represented by the following structural formula:

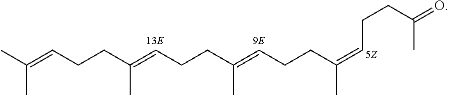 [SMILES: CC(=O)CC/C=C(/C)CC/C=C(\C)CC/C=C(\C)CCC=C(C)C]

The all-trans form can also be synthesized in accordance with a method described in, for example, Bull. Korean Chem. Soc., 2009, Vol. 30, No. 9, 215-217. This literature describes, for example, the method shown by the following synthesis scheme:

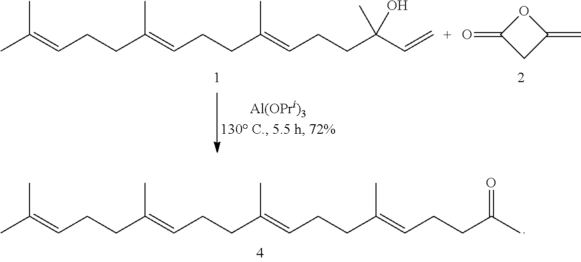

Specifically, in the above reaction formula, geranyllinalool 1 is mixed with Compound 2 and aluminum isopropoxide, and the mixture is gradually heated to 130° C. to allow the reaction to occur. After the completion of the reaction, the residue Compound 2 is removed and the reaction mixture is diluted with 5% sodium carbonate so that the residue aluminum propoxide is quenched. In this way, the all-trans form can be obtained. The obtained all-trans form is subsequently purified by, for example, silica gel chromatography using dichloromethane as an eluent.

(3) Mixtures of all-Trans Form and 5Z-Mono-Cis Form

Mixtures of the all-trans form and the 5Z-mono-cis form can be obtained by adding the all-trans form or the 5Z-mono-cis form to a marketed teprenone.

### GGA Content

The GGA content of the ophthalmic composition is preferably 0.00001% by weight or more, more preferably 0.0001% by weight or more, further more preferably 0.001% by weight or more, relative to the total amount of the composition. The GGA content may be 0.01% by weight or more, 0.1% by weight or more, or 1% by weight or more. The GGA in the above ranges is sufficient to exert its pharmacological action.

The GGA content of the ophthalmic composition is preferably 10% by weight or less, more preferably 5% by weight or less, further more preferably 3% by weight or less, relative to the total amount of the composition. The ophthalmic composition comprising GGA in the above ranges allows clearer vision and hardly causes blurred vision.

The GGA content of the ophthalmic composition is, for example, about 0.00001 to 10% by weight, about 0.00001 to 5% by weight, about 0.00001 to 3% by weight, about 0.0001 to 10% by weight, about 0.0001 to 5% by weight, about 0.0001 to 3% by weight, about 0.001 to 10% by weight, about 0.001 to 5% by weight, about 0.001 to 3% by weight, about 0.01 to 10% by weight, about 0.01 to 5% by weight, about 0.01 to 3% by weight, about 0.1 to 10% by weight, about 0.1 to 5% by weight, about 0.1 to 3% by weight, about 1 to 10% by weight, about 1 to 5% by weight, or about 1 to 3% by weight, relative to the total amount of the composition.

### Preparations

The form of the ophthalmic composition may be a liquid, a fluid, a gel or a semi-solid. Generally, components in a liquid or fluid composition tend to be adsorbed to a container wall. Hence, the present invention is suitably applied to a liquid or fluid ophthalmic composition. In addition, GGA in an aqueous composition tends to be adsorbed to a container wall and thus the present invention is also suitably applied to an aqueous composition.

The type of the ophthalmic composition is not particularly limited. Examples thereof include an eye drop, an eye wash, a contact lens-wearing solution, a contact lens solution (e.g., a washing solution, a storage solution, a sterilizing solution, a multipurpose solution, a package solution, etc.), a preservative for a harvested ocular tissue (a cornea etc.) for transplantation, an irrigating solution for surgery, an ophthalmic ointment (e.g., a water-soluble ophthalmic ointment, an oil-soluble ophthalmic ointment, etc.), an intraocular injection (e.g., an intravitreal injection), etc. Among these, preferred are an eye drop, an eye wash, an ophthalmic ointment and an intraocular injection.

Preparation methods for an ophthalmic preparation are well known. An ophthalmic preparation can be prepared by mixing GGA with a pharmaceutically acceptable base or carrier, and as needed a pharmaceutically acceptable additive for an ophthalmic preparation and another active ingredient (a physiologically or pharmacologically active component).

### <Bases or Carriers>

Examples of the base or carrier include water; an aqueous solvent such as a polar solvent; a polyalcohol; a vegetable oil; and an oily base. Examples of the base or carrier for an intraocular injection include water for injection and physiological saline.

These bases or carriers can be used alone or in combination of two or more thereof.

### <Additives>

Examples of the additive include a surfactant, a flavor or cooling agent, an antiseptic, a bactericide or antibacterial agent, a pH adjusting agent, a tonicity agent, a chelating agent, a buffering agent, a stabilizer, an antioxidant, and a thickening agent. An intraocular injection may contain a solubilizing agent, a suspending agent, a tonicity agent, a buffering agent, a soothing agent, a stabilizer, and an antiseptic.

These additives can be used alone or in combination of two or more thereof.

The additives will be exemplified below.

Surfactants: for example, nonionic surfactants such as polyoxyethylene (hereinafter sometimes referred to as “POE”)-polyoxypropylene (hereinafter sometimes referred to as “POP”) block copolymers (e.g., poloxamer 407, poloxamer 235, poloxamer 188), ethylenediamine POE-POP block copolymer adducts (e.g., poloxamine), POE sorbitan fatty acid esters (e.g., polysorbate 20, polysorbate 60, polysorbate 80 (TO-10 etc.)), POE hydrogenated castor oils (e.g., POE (60) hydrogenated castor oil (HCO-60 etc.)), POE castor oils, POE alkyl ethers (e.g., polyoxyethylene (9) lauryl ether, polyoxyethylene (20) polyoxypropylene (4) cetyl ether), and polyoxyl stearate;

amphoteric surfactants such as glycine-type amphoteric surfactants (e.g., alkyl diaminoethyl glycine, alkyl polyaminoethyl glycine), betaine-type amphoteric surfactants (e.g., lauryldimethylaminoacetic betaine, imidazolinium betaine);
cationic surfactants such as alkyl quaternary ammonium salts (e.g., benzalkonium chloride, benzethonium chloride); etc.

The numbers in the parentheses represent the molar number of added POE or POP.

Flavors or cooling agents: for example, camphor, borneol, terpenes (these may be in the d-form, l-form, or dl-form); essential oils such as mentha water, eucalyptus oil, bergamot oil, anethole, eugenol, geraniol, menthol, limonene, mentha oil, peppermint oil, rose oil, etc.

Antiseptics, bactericides, or antibacterial agents: for example, polidronium chloride, alkyldiaminoethylglycine hydrochloride, sodium benzoate, ethanol, benzalkonium chloride, benzethonium chloride, chlorhexidine gluconate, chlorobutanol, sorbic acid, potassium sorbate, sodium dehydroacetate, methyl paraoxybenzoate, ethyl paraoxybenzoate, propyl paraoxybenzoate, butyl paraoxybenzoate, oxyquinoline sulfate, phenethyl alcohol, benzyl alcohol, biguanide compounds (in particular, polyhexamethylene biguanide or its hydrochloride etc.), Glokill (Rhodia Ltd.), etc.

pH adjusting agents: for example, hydrochloric acid, sodium hydroxide, potassium hydroxide, calcium hydroxide, magnesium hydroxide, triethanolamine, monoethanolamine, diisopropanolamine, sulfuric acid, phosphoric acid, etc.

Tonicity agents: for example, sodium bisulfite, sodium sulfite, potassium chloride, calcium chloride, sodium chloride, magnesium chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, glycerin, propylene glycol, etc.

Chelating agents: for example, ascorbic acid, edetic acid tetrasodium, sodium edetate, citric acid, etc.

Buffering agents: for example, phosphate buffering agents; citrate buffering agents such as citric acid and sodium citrate; acetate buffering agents such as acetic acid, potassium acetate, and sodium acetate; carbonate buffering agents such as sodium bicarbonate and sodium carbonate; borate buffering agents such as boric acid and borax; amino acid buffering agents such as taurine, aspartic acid and its salts (e.g., potassium salts etc.), and ε-aminocaproic acid; etc.

Among the above, phosphate buffering agents are preferred for pH adjustment and the use of a phosphate buffering agent reduces adsorption of GGA to a container wall, thereby further effectively reducing the loss of the GGA content of the composition. The use of a phosphate buffering agent also reduces white turbidity during storage at low temperature, reduces adsorption of GGA to a contact lens, and improves the thermal and light stabilities.

The phosphate buffering agents can be used alone or in combination of two or more thereof.

The phosphate buffering agent is not particularly limited and examples thereof include phosphoric acid; alkali metal phosphates such as disodium hydrogen phosphate, sodium dihydrogen phosphate, trisodium phosphate, dipotassium hydrogen phosphate, potassium dihydrogen phosphate, and tripotassium phosphate; alkaline earth metal phosphates such as calcium phosphate, calcium hydrogen phosphate, calcium dihydrogen phosphate, monomagnesium phosphate, dimagnesium phosphate (magnesium hydrogen phosphate), and trimagnesium phosphate; and ammonium phosphates such as diammonium hydrogen phosphate and ammonium dihydrogen phosphate. The phosphate buffering agent may be an anhydride or hydrate.

Among the above, preferably at least one selected from the group consisting of phosphoric acid and alkali metal phosphates is used, and more preferably at least one selected from the group consisting of phosphoric acid and sodium phosphates is used.

Preferred combinations of phosphate buffering agents are, for example, a combination of phosphoric acid, disodium hydrogen phosphate, sodium dihydrogen phosphate, and trisodium phosphate; a combination of phosphoric acid, disodium hydrogen phosphate, and sodium dihydrogen phosphate; a combination of phosphoric acid, disodium hydrogen phosphate, and trisodium phosphate; a combination of phosphoric acid, sodium dihydrogen phosphate, and trisodium phosphate; a combination of disodium hydrogen phosphate, sodium dihydrogen phosphate, and trisodium phosphate; a combination of phosphoric acid and disodium hydrogen phosphate; a combination of phosphoric acid and sodium dihydrogen phosphate; a combination of phosphoric acid and trisodium phosphate; a combination of disodium hydrogen phosphate and sodium dihydrogen phosphate; a combination of disodium hydrogen phosphate and trisodium phosphate; and a combination of sodium dihydrogen phosphate and trisodium phosphate.

Among these, preferred are a combination of phosphoric acid, disodium hydrogen phosphate, and sodium dihydrogen phosphate; a combination of phosphoric acid and disodium hydrogen phosphate; a combination of phosphoric acid and sodium dihydrogen phosphate; and a combination of disodium hydrogen phosphate and sodium dihydrogen phosphate. More preferred is a combination of disodium hydrogen phosphate and sodium dihydrogen phosphate.

The phosphate buffering agent content expressed in terms of a corresponding anhydride is preferably 0.001% by weight or more, more preferably 0.005% by weight or more, further more preferably 0.01% by weight or more, further more preferably 0.05% by weight or more, relative to the total amount of the composition. The phosphate buffering agent in the above ranges is sufficient to exhibit the effects of stabilizing GGA, reducing white turbidity at low temperature, and reducing adsorption of GGA to a container wall or a contact lens.

The phosphate buffering agent content is preferably 10% by weight or less, more preferably 7% by weight or less, further more preferably 5% by weight or less, further more preferably 3% by weight or less, relative to the total amount of the composition. When GGA is in the above ranges, the ophthalmic composition exhibits reduced eye irritancy.

The phosphate buffering agent content expressed in terms of a corresponding anhydride is, for example, about 0.001 to 10% by weight, about 0.001 to 7% by weight, about 0.001 to 5% by weight, about 0.001 to 3% by weight, about 0.005 to 10% by weight, about 0.005 to 7% by weight, about 0.005 to 5% by weight, about 0.005 to 3% by weight, about 0.01 to 10% by weight, about 0.01 to 7% by weight, about 0.01 to 5% by weight, about 0.01 to 3% by weight, about 0.05 to 10% by weight, about 0.05 to 7% by weight, about 0.05 to 5% by weight, or about 0.05 to 3% by weight, relative to the total amount of the ophthalmic composition.

The phosphate buffering agent content is preferably 0.0005 parts by weight or more, more preferably 0.001 parts by weight or more, further more preferably 0.005 parts by weight or more, further more preferably 0.01 parts by weight or more, relative to 1 part by weight of GGA. The phosphate buffering agent in the above ranges is sufficient to exhibit the effects of stabilizing GGA, reducing white turbidity at low temperature, and reducing adsorption of GGA to a container wall or a contact lens.

The phosphate buffering agent content is preferably 5000 parts by weight or less, more preferably 1000 parts by weight or less, further more preferably 500 parts by weight or less, further more preferably 200 parts by weight or less, relative to 1 part by weight of GGA. When the phosphate buffering agent is in the above ranges, the ophthalmic composition exhibits reduced eye irritancy.

The phosphate buffering agent content expressed in terms of a corresponding anhydride is, for example, about 0.0005 to 5000 parts by weight, about 0.0005 to 1000 parts by weight, about 0.0005 to 500 parts by weight, about 0.0005 to 200 parts by weight, about 0.001 to 5000 parts by weight, about 0.001 to 1000 parts by weight, about 0.001 to 500 parts by weight, about 0.001 to 200 parts by weight, about 0.005 to 5000 parts by weight, about 0.005 to 1000 parts by weight, about 0.005 to 500 parts by weight, about 0.005 to 200 parts by weight, about 0.01 to 5000 parts by weight, about 0.01 to 1000 parts by weight, about 0.01 to 500 parts by weight, or about 0.01 to 200 parts by weight, relative to 1 part by weight of GGA.

Stabilizers: for example, trometamol, sodium formaldehyde sulfoxylate (rongalit), tocopherol, sodium pyrosulfite, monoethanolamine, aluminum monostearate, glyceryl monostearate, etc.

Antioxidants: for example, water-soluble antioxidants such as ascorbic acid, ascorbic acid derivatives (ascorbic acid-2-sulfate disodium salts, sodium ascorbate, ascorbic acid-2-magnesium phosphate, ascorbic acid-2-sodium phosphate, etc.), sodium bisulfite, sodium sulfite, sodium thiosulfate, etc.

The ophthalmic composition used in the present invention may comprise a fat-soluble antioxidant and the use of a fat-soluble antioxidant reduces adsorption of the ophthalmic composition to a container wall, thereby further effectively reducing the loss of the GGA content of the composition. The use of a fat-soluble antioxidant also further effectively reduces adsorption of GGA to a contact lens, and further effectively improves the thermal and light stabilities of GGA.

Examples of the fat-soluble antioxidant include butyl group-containing phenols such as butylhydroxytoluene (BHT) and butylhydroxyanisole (BHA); nordihydroguaiaretic acid (NDGA); ascorbic acid esters such as ascorbyl palmitate, ascorbyl stearate, ascorbyl aminopropyl phosphate, ascorbyl tocopherol phosphate, ascorbic acid triphosphate, and ascorbyl palmitate phosphate; tocopherols such as α-tocopherol, β-tocopherol, γ-tocopherol, and δ-tocopherol; tocopherol derivatives such as tocopherol acetate, tocopherol nicotinate, and tocopherol succinate; gallic acid esters such as ethyl gallate, propyl gallate, octyl gallate, and dodecyl gallate; propyl gallate; 3-butyl-4-hydroxyquinolin-2-one; vegetable oils such as soybean oil, rapeseed oil, olive oil, and sesame oil; carotenoids such as lutein and astaxanthin; polyphenols such as anthocyanins, catechin, tannin, and curcumin; the vitamin A group including retinol, retinol esters (retinol acetate, retinol propionate, retinol butyrate, retinol octylate, retinol laurate, retinol stearate, retinol myristate, retinol oleate, retinal linolenate, retinol linoleate, retinol palmitate, etc.), retinal, retinal esters (retinal acetate, retinal propionate, retinal palmitate, etc.), retinoic acid, retinoic acid esters (methyl retinoate, ethyl retinoate, retinol retinoate, tocopheryl retinoate, etc.), dehydro forms of retinol, dehydro forms of retinal, dehydro forms of retinoic acid, provitamin A (α-carotene, β-carotene, γ-carotene, δ-carotene, lycopene, zeaxanthin, β-cryptoxanthin, echinenone, etc.), and vitamin A; CoQ10, etc. These compounds are marketed.

Among these, preferred are butyl group-containing phenols, NDGA, ascorbic acid esters, tocopherols, tocopherol derivatives, gallic acid esters, propyl gallate, and 3-butyl-4-hydroxyquinolin-2-one, vegetable oils, and the vitamin A group. Among these, preferred are butyl group-containing phenols, tocopherols, tocopherol derivatives, vegetable oils, and the vitamin A group, more preferred are butyl group-containing phenols, vegetable oils, retinol, and retinal esters, and furthermore preferred are BHT, BHA, sesame oil, and retinal palmitate.

These fat-soluble antioxidants can be used alone or in combination of two or more thereof.

The fat-soluble antioxidant content of the ophthalmic composition is preferably 0.00001% by weight or more, more preferably 0.00005% by weight or more, furthermore preferably 0.0001% by weight or more, further more preferably 0.0005% by weight or more, relative to the total amount of the ophthalmic composition. The fat-soluble antioxidant in the above ranges is sufficient to exhibit the effects of reducing adsorption of GGA to a container wall (thereby reducing the loss of the GGA content), reducing adsorption of GGA to a contact lens, and improving the thermal and light stabilities of GGA.

The fat-soluble antioxidant content of the ophthalmic composition is preferably 10% by weight or less, more preferably 5% by weight or less, further more preferably 2% by weight or less, further more preferably 1% by weight or less, relative to the total amount of the composition. When the fat-soluble antioxidant is in the above ranges, the ophthalmic composition exhibits reduced eye irritancy.

The fat-soluble antioxidant content of the ophthalmic composition is, for example, about 0.00001 to 10% by weight, about 0.00001 to 5% by weight, about 0.00001 to 2% by weight, about 0.00001 to 1% by weight, about 0.00005 to 10% by weight, about 0.00005 to 5% by weight, about 0.00005 to 2% by weight, about 0.00005 to 1% by weight, about 0.0001 to 10% by weight, about 0.0001 to 5% by weight, about 0.0001 to 2% by weight, about 0.0001 to 1% by weight, about 0.0005 to 10% by weight, about 0.0005 to 5% by weight, about 0.0005 to 2% by weight, or about 0.0005 to 1% by weight, relative to the total amount of the ophthalmic composition.

The fat-soluble antioxidant content of the ophthalmic composition is preferably 0.0001 parts by weight or more, more preferably 0.001 parts by weight or more, further more preferably 0.005 parts by weight or more, further more preferably 0.01 parts by weight or more, relative to 1 part by weight of GGA. The fat-soluble antioxidant in the above ranges is sufficient to exhibit the effects of reducing adsorption of GGA to a container wall (thereby reducing the loss of the GGA content), reducing adsorption of GGA to a contact lens, and improving the thermal and light stabilities of GGA.

The fat-soluble antioxidant content of the ophthalmic composition is preferably 100 parts by weight or less, more preferably 50 parts by weight or less, furthermore preferably 10 parts by weight or less, further more preferably 5 parts by weight or less, relative to 1 part by weight of GGA. When the fat-soluble antioxidant is in the above ranges, the ophthalmic composition exhibits reduced eye irritancy.

The fat-soluble antioxidant content of the ophthalmic agent is, for example, about 0.0001 to 100 parts by weight, about 0.0001 to 50 parts by weight, about 0.0001 to 10 parts by weight, about 0.0001 to 5 parts by weight, about 0.001 to 100 parts by weight, about 0.001 to 50 parts by weight, about 0.001 to 10 parts by weight, about 0.001 to 5 parts by weight, about 0.005 to 100 parts by weight, about 0.005 to 50 parts by weight, about 0.005 to 10 parts by weight, about 0.005 to 5 parts by weight, about 0.01 to 100 parts by weight, about 0.01 to 50 parts by weight, about 0.01 to 10 parts by weight, or about 0.01 to 5 parts by weight, relative to 1 part by weight of GGA.

Thickening agents: for example, guar gum; hydroxypropyl guar gum; high molecular cellulose compounds such as methylcellulose, ethylcellulose, hydroxypropyl methylcellulose, hydroxyethyl cellulose, and carboxymethyl cellulose sodium; gum arabic; karaya gum; xanthan gum; agar-agar; alginic acid; a-cyclodextrin; dextrin; dextran; heparin; heparinoid; heparin sulfate; heparan sulfate; hyaluronic acid; hyaluronates (sodium salts etc.); sodium chondroitin sulfate; starch; chitin and its derivatives; chitosan and its derivatives; carrageenan; sorbitol; high molecular polyvinyl compounds such as polyvinylpyrrolidone, polyvinyl alcohol, and polyvinyl methacrylate; carboxy vinyl polymers such as alkali metal polyacrylates (sodium salts, potassium salts, etc.), amine polyacrylates (monoethanolamine salts, diethanolamine salts, triethanolamine salts, etc.), and ammonium polyacrylates; casein; gelatin; collagen; pectin; elastin; ceramide; liquid paraffin; glycerin; polyethylene glycol; macrogol; polyethyleneimine alginates (sodium salts etc.); alginate esters (propylene glycol esters etc.); powdered tragacanth; triisopropanolamine; etc.

### <Other Pharmacologically or Physiologically Active Components>

Pharmacologically or physiologically active components other than GGA can be used alone or in combination of two or more thereof.

Examples of the pharmacologically or physiologically active components include prophylactic or therapeutic components for a retinal disease, nerve growth factors, decongestants, drugs for restoring extraocular muscle function, anti-inflammatory drugs or astringent drugs, antihistaminics or antiallergics, vitamins, amino acids, antibacterial drugs or bactericides, sugars, high molecular compounds, celluloses or their derivatives, local anesthetics, etc. These components will be exemplified below.

Prophylactic or therapeutic components for a retinal disease: for example, prostaglandin F2α derivatives such as prost drugs (latanoprost, travoprost, tafluprost, etc.), prostamide drugs (bimatoprost etc.) and prostone drugs (isopropyl unoprostone); sympatholytic drugs such as β-blockers (timolol maleate, gel-forming timolol, carteolol hydrochloride, gel-forming carteolol, etc.), β1-blockers (betaxolol hydrochloride etc.), αβ-blockers (levobunolol hydrochloride, nipradilol, bunazosin hydrochloride, etc.) and α2 blockers (brimonidine tartrate); parasympathomimetic drugs such as pilocarpine hydrochloride and distigmine bromide; sympathomimetic drugs such as epinephrine, epinephrine hydrogen tartrate and dipivefrin hydrochloride; carbonic anhydrase inhibitors such as dorzolamide hydrochloride and brinzolamide; specific inhibitors to ROCK (Rho-associated coiled coil forming protein kinase) such as SNJ-1656 and K-115; calcium antagonists such as lomerizine hydrochloride; EP2 agonists such as DE-117; adenosine A2a receptor agonists such as OPA-6566; therapeutic agents for age-related macular degeneration such as VEGF aptamers (pegaptanib sodium) and VEGF inhibitors (ranibizumab, bevacizumab); etc.

Nerve growth factors: for example, nerve growth factor (NGF), brain-derived nerve growth factor (BDNF), glial cell line-derived neurotrophic factor (GDNF), etc.

Since nutritional factors including nerve growth factors are contained in serum, it is possible to add serum from a patient to a preparation for the patient.

Decongestants: for example, α-adrenergic agonists such as epinephrine, epinephrine hydrochloride, ephedrine hydrochloride, oxymetazoline hydrochloride, tetrahydrozoline hydrochloride, naphazoline hydrochloride, phenylephrine hydrochloride, methylephedrine hydrochloride, epinephrine hydrogen tartrate, naphazoline nitrate, etc. These may be in the d-form, l-form, or dl-form.

Drugs for restoring extraocular muscle function: for example, cholinesterase inhibitors having an active center similar to that of acetylcholine, such as neostigmine methylsulfate, tropicamide, helenien, atropine sulfate, etc.

Anti-inflammatory drugs or astringent drugs: for example, zinc sulfate, zinc lactate, allantoin, ε-aminocaproic acid, indomethacin, lysozyme chloride, silver nitrate, pranoprofen, azulene sulfonate sodium, dipotassium glycyrrhizinate, diammonium glycyrrhizinate, diclofenac sodium, bromfenac sodium, berberine chloride, berberine sulfate, etc.

Antihistaminics or antiallergics: for example, acitazanolast, diphenhydramine or its salts (hydrochloride etc.), chlorpheniramine maleate, ketotifen fumarate, levocabastine or its salts (hydrochloride etc.), amlexanox, ibudilast, tazanolast, tranilast, oxatomide, suplatast or its salts (tosilate etc.), sodium cromoglicate, pemirolast potassium, etc.

Vitamins: for example, retinol acetate, retinol palmitate, pyridoxine hydrochloride, flavin adenine dinucleotide sodium, pyridoxal phosphate, cyanocobalamin, panthenol, calcium pantothenate, sodium pantothenate, ascorbic acid, tocopherol acetate, tocopherol nicotinate, tocopherol succinate, tocopherol calcium succinate, ubiquinone derivatives, etc.

Amino acids: for example, aminoethylsulfonic acid (taurine), glutamic acid, creatinine, sodium aspartate, potassium aspartate, magnesium aspartate, magnesium potassium aspartate, sodium glutamate, magnesium glutamate, ε-aminocaproic acid, glycine, alanine, arginine, lysine, γ-aminobutyric acid, γ-aminovaleric acid, sodium chondroitin sulfate, etc. These may be in the d-form, l-form, or dl-form.

Antibacterial drugs or bactericides: for example, alkylpolyaminoethylglycine, chloramphenicol, sulfamethoxazole, sulfisoxazole, sulfamethoxazole sodium, sulfisoxazole diethanolamine, sulfisoxazole monoethanolamine, sulfisomezole sodium, sulfisomidine sodium, ofloxacin, norfloxacin, levofloxacin, lomefloxacin hydrochloride, acyclovir, etc.

Sugars: for example, monosaccharides, disaccharide, in particular, glucose, maltose, trehalose, sucrose, cyclodextrin, xylitol, sorbitol, mannitol, etc.

High molecular compounds: for example, alginic acid, sodium alginate, dextrin, dextran, pectin, hyaluronic acid, chondroitin sulfate, (completely or partially saponified) polyvinyl alcohol, polyvinylpyrrolidone, carboxy vinyl polymers, macrogol, pharmaceutically acceptable salts thereof, etc.

Celluloses or their derivatives: for example, ethylcellulose, hydroxyethyl cellulose, hydroxypropyl cellulose, hydroxypropyl methylcellulose, methylcellulose, carboxymethyl cellulose, carboxymethylcellulose sodium, carboxyethyl cellulose, nitrocellulose, etc.

Local anesthetics: for example, chlorobutanol, procaine hydrochloride, lidocaine hydrochloride, etc.

pH

The pH of the ophthalmic preparation is preferably 4 or higher, more preferably 5.5 or higher, further more preferably 6 or higher, further more preferably 6.5 or higher. The preparation having a pH value in the above ranges is excellent in the thermal and light stabilities of GGA.

The pH of the ophthalmic preparation is preferably 9 or lower, more preferably 8.5 or lower, further more preferably 8 or lower, further more preferably 7.5 or lower. The ophthalmic preparation having a pH value in the above ranges exhibits reduced eye irritancy.

### Ophthalmic Container

The ophthalmic container employed in the present invention is an ophthalmic container whose surface in contact with the ophthalmic composition is at least partially or wholly made of at least one material selected from the group consisting of a polyolefin, an acrylic acid resin, a terephthalic acid ester, a 2,6-naphthalene dicarboxylic acid ester, a polycarbonate, a polymethylterpene, a fluorine resin, a polyvinyl chloride, a polyamide, an ABS resin, an AS resin, a polyacetal, a modified polyphenylene ether, a polyarylate, a polysulfone, a polyimide, a cellulose acetate, a hydrocarbon optionally substituted with a halogen atom, an aluminum and a glass.

Examples of the polyolefin include polyethylenes (including high density polyethylene, low density polyethylene, ultra low density polyethylene, linear low density polyethylene, ultra high molecular weight polyethylene, etc.), polypropylenes (including isotactic polypropylene, syndiotactic polypropylene, atactic polypropylene, etc.), ethylene-propylene copolymers, etc.

Examples of the acrylic acid resin include acrylic acid esters such as methyl acrylate, methacrylic acid esters such as methyl methacrylate, cyclohexyl methacrylate and t-butyl cyclohexyl methacrylate, etc.

Examples of the terephthalic acid ester include polyethylene terephthalate, polytrimethylene terephthalate, polybutylene terephthalate, etc.

Examples of the 2,6-naphthalene dicarboxylic acid ester include polyethylene naphthalate, polybutylene naphthalate, etc.

Examples of the fluorine resin include fluorine-substituted polyethylenes (polytetrafluoroethylene, polychlorotrifluoroethylene, etc.), polyvinylidene fluoride, polyvinyl fluoride, perfluoroalkoxy fluorine resins, tetrafluoroethylene-hexafluoropropylene copolymers, ethylene-tetrafluoroethylene copolymers, ethylene-chlorotrifluoroethylene copolymers, etc.

Examples of the polyamide include nylon etc.

Examples of the polyacetal include polyacetals consisting of oxymethylene units, polyacetals containing oxyethylene units, etc.

Examples of the modified polyphenylene ether include polystyrene-modified polyphenylene ether etc.

Examples of the polyarylate include amorphous polyarylate etc.

Examples of the polyimide include aromatic polyimides such as the one obtained by polymerizing pyromellitic dianhydride and 4,4′-diaminodiphenyl ether.

Examples of the cellulose acetate include cellulose diacetate, cellulose triacetate, etc.

Examples of the hydrocarbon optionally substituted with a halogen atom include hydrocarbons such as methane, ethane, propane, butane, ethylene, propylene, 1-butene, 2-butene and 1,3-butadiene; hydrocarbons substituted with a fluorine atom such as fluoromethane, difluoromethane, fluoroform, tetrafluoromethane, 1,1-difluoroethane, 1,2-difluoroethane, 1-fluoropropane, 2-fluoropropane, 1,2-fluoropropane, 1,3-fluoropropane, 1-fluorobutane, 2-fluorobutane, vinyl fluoride, 1,1-difluoroethylene, 1,2-difluoroethylene, trifluoroethylene, tetrafluoroethylene, 3-fluoropropene, 1,3-fluoropropene, 1,1,4,4-tetrafluorobutadiene and perfluorobutadiene; hydrocarbons substituted with a chlorine atom such as chloromethane, dichloromethane, chloroform, tetrachloromethane, 1,1-dichloroethane, 1,2-dichloroethane, 1-chloropropane, 2-chloropropane, 1,2-chloropropane, 1,3-chloropropane, 1-chlorobutane, 2-chlorobutane, vinyl chloride, 1,1-dichloroethylene, 1,2-dichloroethylene, trichloroethylene, tetrachloroethylene, 3-chloropropene, 1,3-chloropropene, 1,1,4,4-tetrachlorobutadiene and perchlorobutadiene; hydrocarbons substituted with a bromine atom such as bromomethane, dibromomethane, bromoform, tetrabromomethane, 1,1-dibromoethane, 1,2-dibromoethane, 1-bromopropane, 2-bromopropane, 1,2-bromopropane, 1,3-bromopropane, 1-bromobutane, 2-bromobutane, vinyl bromide, 1,1-dibromoethylene, 1,2-dibromoethylene, tribromoethylene, tetrabromoethylene, 3-bromopropene, 1,3-bromopropene, 1,1,4,4-tetrabromobutadiene and perbromobutadiene; hydrocarbons substituted with an iodine atom such as iodomethane, diiodomethane, iodoform, tetraiodomethane, 1,1-diiodoethane, 1,2-diiodoethane, 1-iodopropane, 2-iodopropane, 1,2-iodopropane, 1,3-iodopropane, 1-iodobutane, 2-iodobutane, vinyl iodide, 1,1-diiodoethylene, 1,2-diiodoethylene, triiodoethylene, tetraiodoethylene, 3-iodopropene, 1,3-iodopropene, 1,1,4,4-tetraiodobutadiene and periodobutadiene; etc.

Among these, preferred are hydrocarbons and hydrocarbons substituted with a fluorine atom, more preferred are hydrocarbons substituted with a fluorine atom, further more preferred are fluoromethane, difluoromethane, fluoroform and tetrafluoromethane.

Preferably, the hydrocarbon optionally substituted with a halogen atom is in the form of a polymer film formed on part or the whole of the surface in contact with the ophthalmic composition.

The hardness of the polymer film measured by continuous stiffness measurement technique is preferably 0.01 to 5 Gpa, more preferably 0.1 to 1 Gpa. The polymer film having a hardness value in the above ranges possesses flexibility and the effect of reducing adsorption of GGA. Therefore, a container having such a polymer film formed thereon can be suitably used as an ophthalmic container.

Measuring methods for hardness by continuous stiffness measurement technique are well known. Specifically, the hardness is measured as follows.

(1) A small piece (5×5 mm) of the part having the polymer film formed thereon is cut out from the container to be measured and used as a sample. Alternatively, a small piece of a silicon wafer on which a polymer film has been formed under the same conditions as those for the polymer film on the container may be used as a sample because such a polymer film formed on a silicon wafer possesses similar film thickness and hardness to those of the polymer film on the container.
(2) The back side of the sample polymer film is fixed to a sample table made of aluminum with an adhesive.
(3) The measurement is performed using Nano Indenter XP (MTS Nano Instruments) under the following conditions.

Indenter: use a Bercovich indenter with a diamond tip with a radius of curvature of 50 to 100 nm.

Indenter load: set at 2 gf.

Indentation depth: set at 500 nm.

Measurement point depth: measure the hardness at locations with indentation depths of 20 to 40 nm.

Determination of surface position: the indenter is lowered to the surface of the polymer film at a rate of 5 nm/second and the position that gives a stiffness (stiffness parameter) of 125 N/m is determined as the surface of the polymer film.

(4) The measurement of the hardness (Gpa) under the above conditions is repeated at ten or more different measurement points. The intervals between the measurement points need to be 100 μm or more. The thus measured hardness values are averaged to give the hardness (Gpa) of the polymer film.

The film thickness of the above polymer film is not particularly limited but is, for example, 0.02 to 0.5 μm, preferably 0.04 to 0.4 μm, more preferably 0.06 to 0.3 μm. The measurement of the film thickness can be performed using, for example, an ellipsometer (DVA-36L3) manufactured by Mizojiri Optical Co., Ltd.

The method for forming the above polymer film on the surface of a container is not particularly limited and any known method for forming a polymer film can be used. Preferred examples of the method include plasma polymerization performed on a container surface with the use of the above hydrocarbon as a monomer gas. Formation of the polymer film by means of plasma polymerization can provide advantages including the following: a polymer film that is further excellent in the effect of reducing adsorption of GGA and in flexibility can be obtained, film formation can be performed at low temperature, a dense thin film without a pinhole can be formed, and adhesion between the polymer film and a container can be improved.

Preferably, the container material is at least one selected from the group consisting of a polyolefin (in particular a is polyethylene, a polypropylene), a methacrylic acid ester (in particular a methyl methacrylate), a polyethylene terephthalate, a polycarbonate, a polymethylterpene and a fluorine-substituted polyethylene (in particular a polytetrafluoroethylene).

The inner surface of the ophthalmic container may have a layer or film made of the above material. The container itself may be shaped from the above material. At least part of the surface in contact with the ophthalmic composition is made of the above material, and preferably the whole surface in contact with the ophthalmic composition is made of the above material.

In the case of a container having a spout or nozzle, such as an eye drop container, an eye wash container and an ointment container, the whole of the container including the spout or nozzle may be shaped from the above material, or only the body excluding the spout or nozzle may be shaped from the above material. The inner surface of the whole container may have a layer or film made of the above material. Only the inner surface of the body may have a layer or film made of the above material.

In the case of a container whose edge is compression-bonded, such as a container for a package solution for a contact lens, the whole of the container including the compression-bonded part may be made of the above material, or only the body excluding the compression-bonded part may be made of the above material. The inner surface of the whole of the container including the compression-bonded part may have a layer or film made of the above material. Only the inner surface of the body may have a layer or film made of the above material.

The shape, volume, wall thickness, etc. of the container are not particularly limited. According to the type of the container, the shape, volume, wall thickness, etc. are selected from those usually used.

In cases where the inner wall of the container has a layer or film made of the above material, the layer or film may be stacked on the body of the container or formed thereon by vapor deposition, plasma CVD, plasma polymerization, sputtering, etc. The thickness of the layer or film made of the above material is not particularly limited and may be, for example, about 10 nm to 5 mm.

The type of the ophthalmic container can be selected according to the type of the ophthalmic composition. Examples of the ophthalmic container include: for the eye drop, an eye drop container; for the eye wash, an eye wash container; for the contact lens-wearing solution, a container for a contact lens-wearing solution; for the contact lens washing solution, a contact lens washing container; for the contact lens storage solution, a contact lens storage container; for the contact lens sterilizing solution, a contact lens sterilization container; for the contact lens multipurpose solution, a container for a contact lens multipurpose solution; for the contact lens package solution, a container for a contact lens package solution; for the preservative for a harvested ocular tissue, a preservation container for a harvested ocular tissue; for the irrigating solution for surgery, a container for holding an irrigating solution for surgery or a tube for supplying an irrigating solution during surgery; for the ophthalmic ointment, an ophthalmic ointment container; for the intraocular injection, a container for an injection or a syringe; etc. Among these, preferred are an eye drop container, an eye wash container, a container for holding an irrigating solution for surgery or a tube for supplying an irrigating solution during surgery, an ophthalmic ointment container, and a container for an injection or a syringe, more preferred are an eye drop container, an ophthalmic ointment container, and a container for an injection or a syringe.

The ophthalmic composition is held by, encapsulated in, or filled into the ophthalmic container. The term “fill” herein encompasses the case where the container is completely filled with the ophthalmic composition and the case where some air space remains in the container.

The container material according to the present invention, which is at least one selected from the group consisting of a polyolefin, an acrylic acid resin, a terephthalic acid ester, a 2,6-naphthalene dicarboxylic acid ester, a polycarbonate, a polymethylterpene, a fluorine resin, a polyvinyl chloride, a polyamide, an ABS resin, an AS resin, a polyacetal, a modified polyphenylene ether, a polyarylate, a polysulfone, a polyimide, a cellulose acetate, a hydrocarbon optionally substituted with a halogen atom, an aluminum and a glass, may also comprise an additive used in the production of a container or film, for example, a plasticizer, a cross linking agent, a mold releasing agent, a thickening agent, a reinforcing agent, a flame retardant, a light-blocking agent, an ultraviolet absorber, a colorant, an anti-clouding agent, an anti-static agent, a polymerization initiator, an antioxidant, a fungicide, a lubricant, a filler, etc.

### Usage

The usage of the kit of the present invention varies depending on the dosage form of the ophthalmic composition and the route of administration is appropriately selected in accordance with the dosage form of the ophthalmic composition.

For example, when the composition of the present invention is an eye drop, the eye drop comprising GGA in the above concentration ranges is instilled, for example, about 1 to 5 times a day, preferably about 1 to 3 times a day, in an amount of about 1 to 2 drops each time.

When the composition of the present invention is an eye wash, eye washing is performed, for example, about 1 to 10 times a day, preferably about 1 to 5 times a day, each time using about to 20 mL of the eye wash comprising GGA in the above concentration ranges.

When the composition of the present invention is an ophthalmic ointment, the ophthalmic ointment comprising GGA in the above concentration ranges is applied to the eye, for example, about 1 to 5 times a day, preferably about 1 to 3 times a day, in an amount of about 0.001 to 5 g each time.

When the composition of the present invention is an intraocular injection, the intraocular injection comprising GGA in the above concentration ranges is injected, for example, about 1 to 3 times per day to 14 days, preferably once per day to 14 days, in an amount of about 0.005 to 1 mL each time.

When the composition of the present invention is a contact lens solution (a washing solution, a storage solution, a sterilizing solution, a multipurpose solution, package solution, etc.), a preservative for a harvested ocular tissue (a cornea etc.) for transplantation, or an irrigating solution for surgery, such a composition comprising GGA in the above concentration ranges is used in a usual dosage and regimen of such a type of preparation.

The administration period varies depending on the type and stage of the disease, the age, weight, and sex of the patient, the route of administration, etc., and can be selected as appropriate, for example, from the range from about one day to 30 years. When the retinal protective action exhibited by the ophthalmic composition of the present invention suppresses the progress of a retinal disease, the administration can be further continued.

### Others

The present invention includes a method for reducing the loss of the GGA content of an ophthalmic composition and a method for reducing adsorption of GGA to an ophthalmic container, each method comprising employing, as an ophthalmic container for holding the ophthalmic composition comprising GGA, a container whose surface in contact with the ophthalmic composition is at least partially or wholly made of at least one container material selected from the group consisting of a polyolefin, an acrylic acid resin, a terephthalic acid ester, a 2,6-naphthalene dicarboxylic acid ester, a polycarbonate, a polymethylterpene, a fluorine resin, a polyvinyl chloride, a polyamide, an ABS resin, an AS resin, a polyacetal, a modified polyphenylene ether, a polyarylate, a polysulfone, a polyimide, a cellulose acetate, a hydrocarbon optionally substituted with a halogen atom, an aluminum, and glass.

In these methods of the present invention, the components of the ophthalmic composition, the dosage of the composition, the properties of the composition, examples of the container material, the shape of the container, the properties of the container, etc. are as described for the ophthalmic composition kit of the present invention.

### Examples

The present invention will be described in more detail below with reference to Examples, but the present invention is not limited thereto.

### (1) Preparation of Geranylgeranylacetone

Marketed teprenone (all-trans form: 5Z-mono-cis form=6:4 (weight ratio)) (Wako Pure Chemical Industries, Ltd.) was purchased and the all-trans form and the 5Z-mono-cis form were separated and purified by silica gel chromatography.

The above preparative purification was carried out using silica gel (PSQ60B, Fuji Silysia Chemical Ltd.) filled in a glass tube and a mobile phase of n-hexane/ethyl acetate (9:1). After the separation, each fraction was concentrated and dried under reduced pressure and the degrees of purification and structures of the all-trans form and the 5Z-mono-cis form were determined by GC and ¹H-NMR (solvent: deuterated chloroform; internal standard: tetramethylsilane) (about 20% yield).

### <GC Measurement Conditions>

Column: DB-1 (J&W Scientific, 0.53 mm×30 m, film thickness of 1.5 μm)
Column temperature: elevated at a rate of 5° C./minute from 200° C. to 300° C. (10 minutes)
Vaporizing chamber temperature: 280° C.
Detector temperature: 280° C.
Carrier gas: helium
Hydrogen pressure: 60 kPa
Air pressure: 50 kPa
Makeup gas pressure: 75 kPa (nitrogen gas)
Total flow: 41 mL/min
Column flow: 6.52 mL/min
Linear velocity: 58.3 cm/sec
Split ratio: 5:1
Injection volume: 1 μL of 0.1 g/100 mL sample (in ethanol)

### (2) Measurement Method for GGA Concentration

In the examination for the residual ratio of GGA described later, the GGA concentration was measured by the following method.

In accordance with the measurement conditions for the elution test described in PFSB/ELD Notification No. 0412007 “teprenone 100 mg/g fine granule”, the GGA concentration of each eye drop was determined from the area value of the 5Z-mono-cis form (Ac) and the area value of the all-trans form (At) using Japanese pharmacopoeia “teprenone reference standard (all-trans form:5Z-mono-cis form=about 6:4 (weight ratio), Pharmaceutical and Medical Device Regulatory Science Society of Japan)” or teprenone (Wako Pure Chemical Industries) as a reference standard under the HPLC measurement conditions described below. For the eye drop containing teprenone (all-trans form:5Z-mono-cis for m=3:2 (weight ratio)), the GGA content was calculated by summing the amounts of the all-trans form and the 5Z-mono-cis form.

### <HPLC Measurement Conditions>

Detector: ultraviolet absorption spectrometer (measurement wavelength: 210 nm)
Column: YMC-Pack ODS-A (inner diameter: 4.6 mm, length: 15 cm, particle diameter: 3 μm)
Column temperature: 30° C.
Mobile phase: 90% acetonitrile solution
Flow rate: 1.2 to 1.3 mL/min (the 5Z-mono-cis form and the all-trans form are eluted in this order.)
Injection volume: 5 μL of 0.05 g/100 mL sample

### (3) Examination for Residual Ratio of GGA No. 1

Eye drops containing the marketed teprenone or GGA consisting of the all-trans form purified by the above method were prepared as follows. The constitutions of the eye drops are shown in Tables 1 to 3 below.

Specifically, to a surfactant (polysorbate 80 and/or POE castor oil) warmed to 65° C., the teprenone or the all-trans form, and optionally retinol palmitate or sesame oil, were added and dissolved under stirring in a hot water bath at 65° C. for 2 minutes. Water at 65° C. was added and each buffer was added under stirring to give a homogeneous solution. The pH and osmotic pressure were adjusted with hydrochloric acid or sodium hydroxide. This resulting solution was filtered through a membrane filter with a pore size of 0.2 μm (bottle top filter, Thermo Fisher Scientific) to give a clear sterile eye drop. Before the preparation of the sterile eye drops, it was confirmed by HPLC described later that adsorption of GGA to instruments etc., which leads to the loss of the GGA content, did not occur during the preparation procedure.

Five mL of each of the eye drops was poured into various plastic or glass containers (volume: 10 to 15 mL) with a glass volumetric pipette and the containers were sealed. The container materials and the volume are shown in Table 3 below. For these eye drops, the stability test was performed by leaving them to stand in the upright position on a test tube stand at 40° C., 75% RH for 2 hours, 8 hours, and 24 hours. The teprenone or all-trans form content (g/100 mL) in each of the eye drops was quantified under the HPLC conditions described above immediately after the production and after being left to stand for a predetermined period of time, and the residual ratio (%) was calculated.

TABLE 1

| | g/100 mL | | |
|---|---|---|---|
| | Formu- | Formu- | Formu- |
| | lation 1 | lation 2 | lation 3 |
| All-trans form | 0.05 | 0.05 | 0.05 |
| Sodium dihydrogen | 2.00 | 0.30 | — |
| phosphate dihydrate | | | |
| Disodium hydrogen | 0.40 | 3.20 | — |
| phosphate dodecahydrate | | | |
| Boric acid | — | — | 1.30 |
| Borax | — | — | 0.40 |
| Polysorbate 80 | 0.35 | 0.35 | 0.35 |
| Hydrochloric acid | q.s. | q.s. | q.s. |
| Sodium hydroxide | q.s. | q.s. | q.s. |
| Purified water | q.s. | q.s. | q.s. |
| pH | 5.7 | 7.5 | 7.5 |
| Osmotic pressure mOsm | 270 | 260 | 240 |

TABLE 2

g/100 mL

| | Formu- | Formu- | Formu- | Formu- |
|---|---|---|---|---|
| | lation 4 | lation 5 | lation 6 | lation 7 |
| All-trans form | 0.05 | — | 0.05 | — |
| All-trans form: | — | 0.05 | — | 0.05 |
| 5Z-mono-cis form | | | | |
| weight ratio (6:4) | | | | |
| Sodium dihydrogen | 2.000 | 2.000 | — | — |
| phosphate dihydrate | | | | |
| Disodium hydrogen | 0.400 | 0.400 | — | — |
| phosphate | | | | |
| dodecahydrate | | | | |
| Boric acid | — | — | 1.300 | 1.300 |
| Borax | — | — | 0.400 | 0.400 |
| POE castor oil | 0.020 | 0.020 | 0.020 | 0.020 |
| Polysorbate 80 | 0.500 | 0.500 | 0.500 | 0.500 |
| Hydrochloric acid | q.s. | q.s. | q.s. | q.s. |
| Sodium hydroxide | q.s. | q.s. | q.s. | q.s. |
| Purified water | q.s. | q.s. | q.s. | q.s. |
| pH | 5.7 | 5.7 | 7.5 | 7.5 |
| Osmotic pressure | 270 | 270 | 240 | 240 |
| mOsm | | | | |

TABLE 3

| | Formulation | Formulation | Formulation | Formulation | Formulation | Formulation |
|---|---|---|---|---|---|---|
| g/100 mL | 8 | 9 | 10 | 11 | 12 | 13 |
| All-trans form | 0.05 | 0.05 | 0.05 | 0.05 | 0.05 | 0.05 |
| Sodium dihydrogen | 0.30 | — | 0.30 | — | 0.30 | — |
| phosphate dihydrate | | | | | | |
| Disodium hydrogen | 3.20 | — | 3.20 | — | 3.20 | — |
| phosphate dodecahydrate | | | | | | |
| Boric acid | — | 1.30 | — | 1.30 | — | 1.30 |
| Borax | — | 0.40 | — | 0.40 | — | 0.40 |
| POE castor oil | 0.02 | 0.02 | 0.02 | 0.02 | 0.02 | 0.02 |
| Polysorbate 80 | 0.50 | 0.50 | 0.50 | 0.50 | 0.50 | 0.50 |
| Sesame oil | — | — | 0.05 | 0.05 | — | — |
| Retinol palmitate | — | — | — | — | 0.05 | 0.05 |
| Hydrochloric acid | q.s. | q.s. | q.s. | q.s. | q.s. | q.s. |
| Sodium hydroxide | q.s. | q.s. | q.s. | q.s. | q.s. | q.s. |
| Purified water | q.s. | q.s. | q.s. | q.s. | q.s. | q.s. |
| pH | 7.5 | 7.5 | 7.5 | 7.5 | 7.5 | 7.5 |
| Osmotic pressure mOsm | 260 | 240 | 260 | 240 | 260 | 240 |

(Retinol palmitate: 1,700,000 IU/g)

TABLE 4

| | | Volume | | Model |
|---|---|---|---|---|
| Material | Abbr. | and type | Manufacturer | number |
| Glass | GLS | 10 mL centri- | IWAKI | 8084CTF10 |
| | | fuge tube | | |
| Polymethyl- | TPX | 15 mL centri- | Sumitomo | MS-56150 |
| pentene | | fuge tube | Bakelite | |
| Poly- | PC | 10 mL v-bottom | Thermo | 3105-0015 |
| carbonate | | centrifuge tube | Scientific | |
| Polyethylene | PET | 15 mL centri- | CORNING | 430053 |
| terephthalate | | fuge tube | | |
| Poly- | PP | 15 mL conical | Becton, | 352196 |
| propylene | | tube | Dickinson | |
| Polymethyl | PMMA | 10 mL sharp- | Nissui | 06470 |
| methacrylate | | bottom tube P | Pharmaceutical | |
| resin | | | | |
| Low density | LDPE | 10 mL sharp- | As One | 2-467-01 |
| polyethylene | | bottom tube | | |
| Poly | PTFE | 13 mL sharp- | Teflon | FW12413 |
| tetrafluoro- | | bottom test tube | | |
| ethylene | | | | |
| Polystyrene | PS | 15 mL centri- | IWAKI | 2324-015 |
| | | fuge tube | | |
| Polybutylene | PBS | 15 mL centri- | Thermo | 366079 |
| succinate | | fuge tube | Scientific | |

All the containers had a v-bottom. The PC container did not have an attached cap, and accordingly a cap attached to the PP container was used to seal the PC container.

Residual ratio (%)=100×[teprenone or all-trans form content after being left to stand for predetermined period of time (g/100 mL)/teprenone or all-trans form content immediately after production (g/100 mL)]

The results are shown in Tables 5 to 7.

TABLE 5

| Residual ratio | | Example | Example | Example | Example | Example | Example | Comparative | Comparative |
|---|---|---|---|---|---|---|---|---|---|
| (%) | | 1 | 2 | 3 | 4 | 5 | 6 | Example 1 | Example 2 |
| Container material | | GLS | TPX | PC | PET | PP | PMMA | PS | PBS |
| 40° C. | Formulation | 100.2 | 98.1 | 100.3 | 100.2 | 95.4 | 95.0 | 86.8 | 87.8 |
| 2 h | 1 | | | | | | | | |
| | Formulation | 100.1 | 97.7 | 99.4 | 99.7 | 95.0 | 93.3 | 87.3 | 86.4 |
| | 2 | | | | | | | | |
| | Formulation | 99.5 | 97.2 | 99.2 | 99.7 | 93.4 | 91.6 | 82.0 | 81.8 |
| | 3 | | | | | | | | |
| 40° C. | Formulation | 100.1 | 94.3 | 99.6 | 99.9 | 82.3 | 79.2 | 55.7 | 56.4 |
| 8 h | 1 | | | | | | | | |
| | Formulation | 98.9 | 92.9 | 98.6 | 98.9 | 79.2 | 78.0 | 56.2 | 56.9 |
| | 2 | | | | | | | | |
| | Formulation | 98.8 | 92.6 | 99.0 | 98.9 | 76.6 | 74.6 | 46.1 | 46.0 |
| | 3 | | | | | | | | |
| 40° C. | Formulation | 99.4 | 87.0 | 99.4 | 99.6 | 51.1 | 49.6 | 11.2 | 11.4 |
| 24 h | 1 | | | | | | | | |
| | Formulation | 98.4 | 85.9 | 98.2 | 98.9 | 50.0 | 48.5 | 11.7 | 11.7 |
| | 2 | | | | | | | | |
| | Formulation | 98.6 | 85.0 | 98.9 | 98.7 | 45.4 | 43.6 | 7.5 | 7.4 |
| | 3 | | | | | | | | |

TABLE 6

| Residual ratio | | Example | Example | Example | Example | Example | Example | Example | Example | Comparative | Comparative |
|---|---|---|---|---|---|---|---|---|---|---|---|
| (%) | | 7 | 8 | 9 | 10 | 11 | 12 | 13 | 14 | Example 3 | Example 4 |
| Container material | | GLS | TPX | PC | PET | PP | PMMA | LDPE | PTFE | PS | PBS |
| 40° C. | Formulation | 99.3 | 97.8 | 99.2 | 99.1 | 95.3 | 93.3 | 88.9 | 98.6 | 84.9 | 85.5 |
| 2 h | 4 | | | | | | | | | | |
| | Formulation | 100.1 | 97.8 | 99.1 | 100.9 | 95.8 | 94.6 | 89.6 | 99.8 | 85.9 | 86.9 |
| | 5 | | | | | | | | | | |
| | Formulation | 98.6 | 99.0 | 97.3 | 99.2 | 95.0 | 93.3 | 88.7 | 99.0 | 84.1 | 84.7 |
| | 6 | | | | | | | | | | |
| | Formulation | 100.8 | 99.2 | 100.1 | 100.5 | 96.7 | 95.2 | 91.2 | 98.8 | 86.1 | 86.9 |
| | 7 | | | | | | | | | | |
| 40° C. | Formulation | 98.0 | 94.4 | 98.1 | 97.7 | 81.9 | 81.0 | 67.6 | 97.7 | 58.3 | 57.8 |
| 8 h | 4 | | | | | | | | | | |
| | Formulation | 99.7 | 95.9 | 99.0 | 99.2 | 83.9 | 82.2 | 68.0 | 99.6 | 58.5 | 58.8 |
| | 5 | | | | | | | | | | |
| | Formulation | 98.4 | 93.3 | 98.1 | 98.3 | 80.4 | 79.0 | 62.3 | 97.3 | 50.8 | 51.8 |
| | 6 | | | | | | | | | | |
| | Formulation | 100.7 | 96.4 | 100.4 | 99.4 | 82.6 | 82.1 | 64.1 | 98.7 | 51.0 | 52.6 |
| | 7 | | | | | | | | | | |
| 40° C. | Formulation | 100.5 | 92.1 | 100.2 | 100.7 | 59.6 | 59.4 | 35.8 | 100.7 | 16.1 | 16.5 |
| 24 h | 4 | | | | | | | | | | |
| | Formulation | 99.6 | 90.8 | 99.7 | 100.4 | 60.5 | 60.4 | 35.5 | 100.4 | 15.6 | 16.8 |
| | 5 | | | | | | | | | | |
| | Formulation | 100.1 | 89.3 | 100.4 | 99.7 | 53.9 | 54.1 | 26.8 | 100.3 | 8.8 | 9.5 |
| | 6 | | | | | | | | | | |
| | Formulation | 100.3 | 91.0 | 99.9 | 98.9 | 54.5 | 54.4 | 26.8 | 100.6 | 9.0 | 10.5 |
| | 7 | | | | | | | | | | |

TABLE 7

Residual ratio (%)

| | | |
|---|---|---|
| Example | Example | Example |
| 15 | 16 | 17 |

Container material

| | | GLS | PP | LDPE |
|---|---|---|---|---|
| 40° C. | Formu- | 99.6 | 94.7 | 89.6 |
| 2 h | lation 8 | | | |
| | Formu- | 99.4 | 93.9 | 86.5 |
| | lation 9 | | | |
| | Formu- | 99.7 | 96.4 | 92.3 |
| | lation 10 | | | |
| | Formu- | 99.8 | 96.0 | 90.1 |
| | lation 11 | | | |
| | Formu- | 99.5 | 96.1 | 92.3 |
| | lation 12 | | | |
| | Formu- | 100.0 | 95.5 | 90.6 |
| | lation 13 | | | |

As is apparent from Tables 5 to 7, the residual ratio of GGA was very high for the GGA containing-eye drops stored in the containers made of polyethylene, polypropylene, polymethyl methacrylate, polyethylene terephthalate, polycarbonate, polymethylterpene, polytetrafluoroethylene, or glass. On the other hand, the residual ratio of GGA was low for the eye drops stored in the containers made of polystyrene or polybutylene succinate.

The eye drops containing the phosphate buffering agents showed higher residual ratios of GGA as compared with the eye drops containing the borate buffering agents, which is apparent from the comparison between Formulation 2 and Formulation 3 in Table 5, Formulation 8 and Formulation 9 in Table 7, Formulation 10 and Formulation 11 in Table 7, and Formulation 12 and Formulation 13 in Table 7.

The eye drops containing the sesame oil showed improved residual ratios of GGA, which is apparent from the comparison between Formulation 8 and Formulation 10 in Table 5, and Formulation 9 and Formulation 11 in Table 5.

The eye drops containing retinol palmitate showed improved residual ratios of GGA, which is apparent from the comparison between Formulation 8 and Formulation 12 in Table 5, and Formulation 9 and Formulation 13 in Table 5.

### (4) Examination for Residual Ratio of GGA No. 2

Eye drops containing the marketed teprenone, or the all-trans or 5Z-mono-cis form purified by the above method were prepared as follows. The constitutions of the eye drops are shown in Table 8 below.

Specifically, to a surfactant (polysorbate 80 and POE castor oil 60 and/or POE hydrogenated castor oil) warmed to 65° C., the all-transform, teprenone or the 5Z-mono-cis form was added and dissolved under stirring in a hot water bath at 65° C. for 2 minutes. Water at 65° C. was added and each buffer was added under stirring to give a homogeneous solution. The pH and osmotic pressure were adjusted with hydrochloric acid or sodium hydroxide (Formulations 14 to 19).

Five mL of each of Formulations 14 and 15 was poured into various plastic containers (centrifuge tubes) (volume: 10 to 15 mL) with a glass volumetric pipette and the containers were sealed.

Ten mL of each of Formulations 16, 17, 18 and 19 was poured into a 13 mL eye drop container (having the same shape as that of the container for Namida Rohto Dry Eye (trade name) (Rohto Pharmaceutical)) with a glass volumetric pipette in the same manner as above and the containers were sealed. The material and volume of each container is shown in Table 9 below.

For these eye drops, the stability test was performed by leaving them to stand in the upright position at 40° C., 75% RH for 2 hours, 4 hours, 8 hours and 24 hours. The all-trans form, teprenone or 5Z-mono-cis form content (g/100 mL) in each of the eye drops was quantified under the HPLC conditions described above immediately after the production and after being left to stand for a predetermined period of time, and the residual ratio (%) was calculated.

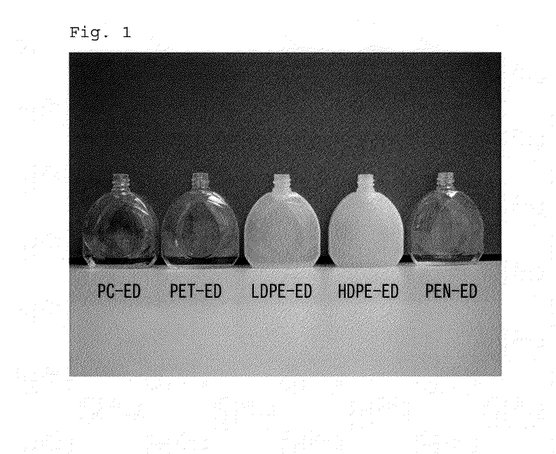

The appearance of the eye drop containers used in this experiment is shown in FIG. 1.

TABLE 8

| | Formulation | Formulation | Formulation | Formulation | Formulation | Formulation |
|---|---|---|---|---|---|---|
| g/100 mL | 14 | 15 | 16 | 17 | 18 | 19 |
| All-trans form | — | — | 1.000 | — | 0.005 | — |
| All-trans form: | | | | | | |
| 5Z-mono-cis form | — | — | — | 1.000 | — | 0.005 |
| weight ratio (6:4) | | | | | | |
| 5Z-mono-cis form | 0.050 | 0.050 | — | — | — | — |
| Sodium dihydrogen | 0.300 | — | — | — | — | — |
| phosphate dihydrate | | | | | | |
| Disodium hydrogen | 3.200 | — | — | — | — | — |
| phosphate | | | | | | |
| dodecahydrate | | | | | | |
| Boric acid | — | 1.300 | 1.300 | 1.300 | 1.300 | 1.300 |
| Borax | — | 0.400 | 0.400 | 0.400 | 0.400 | 0.400 |
| POE castor oil | 0.020 | 0.020 | 0.100 | 0.100 | 0.0005 | 0.0005 |
| POE hydrogenated | — | — | 2.000 | 2.000 | 0.010 | 0.010 |
| castor oil 60 | | | | | | |
| Polysorbate 80 | 0.500 | 0.500 | 2.000 | 2.000 | 0.010 | 0.010 |
| Hydrochloric acid | q.s. | q.s. | q.s. | q.s. | q.s. | q.s. |
| Sodium hydroxide | q.s. | q.s. | q.s. | q.s. | q.s. | q.s. |
| Purified water | q.s. | q.s. | q.s. | q.s. | q.s. | q.s. |
| pH | 7.47 | 7.69 | 7.59 | 7.55 | 7.54 | 7.55 |
| Osmotic pressure | 248 | 236 | 255 | 253 | 236 | 235 |
| mOsm | | | | | | |

TABLE 9

| | | Volume | | Model |
|---|---|---|---|---|
| Material | Abbr. | and type | Manufacturer | number |
| Polycar- | PC | 10 mL v-bottom | Thermo | 3105-0015 |
| bonate | | centrifuge tube | Scientific | |
| Polyethylene | PET | 15 mL centri- | CORNING | 430053 |
| terephthalate | | fuge tube | | |
| Polystyrene | PS | 15 mL centri- | IWAKI | 2324-015 |
| | | fuge tube | | |
| Polybutylene | PBS | 15 mL centri- | Thermo | 366079 |
| succinate | | fuge tube | Scientific | |
| Polycar- | PC-ED | 13 mL eye drop | — | — |
| bonate | | container* | | |
| Polyethylene | PET-ED | 13 mL eye drop | — | — |
| terephthalate | | container* | | |
| Low density | LDPE-ED | 13 mL eye drop | — | — |
| polyethylene | | container* | | |
| High density | HDPE-ED | 13 mL eye drop | — | — |
| polyethylene | | container* | | |
| Polyethylene | PEN-ED | 13 mL eye drop | — | — |
| naphthalate | | container* | | |

*The eye drop containers other than the PET container were produced for this experiment. The PET eye drop container was the container for Namida Rohto Dry Eye (trade name) (Rohto Pharmaceutical).

The results are shown in Tables 10 and 11.

TABLE 10

| | | Residual ratio (%) | | | |
|---|---|---|---|---|---|
| | | Example | Example | Comparative | Comparative |
| | | 18 | 19 | Example 5 | Example 6 |
| | | Container material | | | |
| | | PC | PET | PS | PBS |
| 40° C. | Formu- | 100.0 | 100.4 | 89.6 | 89.9 |
| 2 h | lation 14 | | | | |
| | Formu- | 99.9 | 100.2 | 86.8 | 86.6 |
| | lation 15 | | | | |
| 40° C. | Formu- | 99.4 | 99.6 | 57.1 | 56.5 |
| 8 h | lation 14 | | | | |
| | Formu- | 100.4 | 100.6 | 49.1 | 49.0 |
| | lation 15 | | | | |
| 40° C. | Formu- | 99.0 | 99.3 | 15.2 | 15.9 |
| 24 h | lation 14 | | | | |
| | Formu- | 99.9 | 99.8 | 12.3 | 12.6 |
| | lation 15 | | | | |

TABLE 11

| Residual ratio | | Exam- | Exam- | Exam- | Exam- | Exam- |
|---|---|---|---|---|---|---|
| (%) | | ple 20 | ple 21 | ple 22 | ple 23 | ple 24 |
| Container | | PC- | PET- | LDPE- | HDPE- | PEN- |
| material | | ED | ED | ED | ED | ED |
| 40° C. | Formulation | 100.2 | 100.3 | 100.5 | 100.8 | 100.1 |
| 2 h | 16 | | | | | |
| | Formulation | 98.5 | 100.8 | 98.8 | 98.9 | 99.4 |
| | 17 | | | | | |
| | Formulation | 96.1 | 98.6 | — | — | 98.8 |
| | 18 | | | | | |
| | Formulation | 97.2 | 97.7 | — | — | 98.9 |
| | 19 | | | | | |
| 40° C. | Formulation | 100.3 | 99.5 | 98.4 | 98.6 | 100.3 |
| 4 h | 16 | | | | | |
| | Formulation | 101.0 | 100.6 | 98.9 | 100.9 | 100.7 |
| | 17 | | | | | |
| | Formulation | 95.2 | 98.4 | — | — | 96.2 |
| | 18 | | | | | |
| | Formulation | 95.8 | 97.7 | — | — | 98.4 |
| | 19 | | | | | |
| 40° C. | Formulation | 101.2 | 102.2 | 98.5 | 99.9 | 101.0 |
| 8 h | 16 | | | | | |
| | Formulation | 99.9 | 99.5 | 96.0 | 97.2 | 99.2 |
| | 17 | | | | | |
| | Formulation | 94.6 | 95.7 | — | — | 98.2 |
| | 18 | | | | | |
| | Formulation | 96.3 | 96.2 | — | — | 96.3 |
| | 19 | | | | | |
| 40° C. | Formulation | 101.0 | 101.1 | 95.6 | 97.9 | 101.0 |
| 24 h | 16 | | | | | |
| | Formulation | 98.7 | 99.2 | 92.9 | 95.2 | 97.6 |
| | 17 | | | | | |
| | Formulation | 94.8 | 98.2 | — | — | 97.3 |
| | 18 | | | | | |
| | Formulation | 93.9 | 97.7 | — | — | 97.3 |
| | 19 | | | | | |

As is apparent from Table 10, the residual ratio of GGA was very high for the GGA containing-eye drops stored in the containers made of polycarbonate or polyethylene terephthalate. On the other hand, the residual ratio of GGA was low for the eye drops stored in the containers made of polystyrene or polybutylene succinate.

As is apparent from Table 11, the residual ratio of GGA was very high for the GGA containing-eye drops stored in the eye drop container-shaped containers made of polycarbonate, polyethylene terephthalate, low density polyethylene, high density polyethylene, or polyethylene naphthalate.

### INDUSTRIAL APPLICABILITY

The ophthalmic composition kit of the present invention, in which the loss of the GGA content of the composition is remarkably reduced, is very useful in practice.

1. An ophthalmic composition kit comprising an ophthalmic container holding an ophthalmic composition comprising geranylgeranylacetone,
the ophthalmic container having a surface in contact with the ophthalmic composition, the surface being at least partially or wholly made of at least one container material selected from the group consisting of a polyolefin, an acrylic acid resin, a terephthalic acid ester, a polycarbonate, a polymethylterpene, a fluorine resin and a glass.

2. The ophthalmic composition kit according to claim 1, wherein the container material is at least one selected from the group consisting of a polyethylene, a polypropylene, a polymethyl methacrylate, a polyethylene terephthalate, a polycarbonate, a polymethylterpene, a polytetrafluoroethylene and a glass.

3. The ophthalmic composition kit according to claim 1, wherein the geranylgeranylacetone content of the ophthalmic composition is 0.00001 to 10% by weight relative to the total amount of the composition.

4. The ophthalmic composition kit according to claim 1, wherein the pH of the ophthalmic composition is from 6 to 8.

5. The ophthalmic composition kit according to claim 1, wherein the ophthalmic composition further comprises a phosphate buffering agent.

6. The ophthalmic composition kit according to claim 1, wherein the ophthalmic composition further comprises a fat-soluble antioxidant.

7. The ophthalmic composition kit according to claim 1, wherein the ophthalmic composition is in the form of a liquid, a fluid or a semi-solid.

8. The ophthalmic composition kit according to claim 1, wherein the ophthalmic composition is an eye drop and the ophthalmic container is an eye drop container.

9. A method for reducing the loss of the geranylgeranylacetone content of an ophthalmic composition,
the method comprising the step of employing, as an ophthalmic container for holding the ophthalmic composition comprising geranylgeranylacetone, a container whose surface in contact with the ophthalmic composition is at least partially or wholly made of at least one container material selected from the group consisting of a polyolefin, an acrylic acid resin, a terephthalic acid ester, a polycarbonate, a polymethylterpene, a fluorine resin and a glass,
thereby reducing the loss of the geranylgeranylacetone content of the ophthalmic composition.

10. A method for reducing adsorption of geranylgeranylacetone to a wall of an ophthalmic container,
the method comprising the step of employing, as an ophthalmic container for holding an ophthalmic composition comprising geranylgeranylacetone, a container whose surface in contact with the ophthalmic composition is at least partially or wholly made of at least one container material selected from the group consisting of a polyolefin, an acrylic acid resin, a terephthalic acid ester, a polycarbonate, a polymethylterpene, a fluorine resin and a glass,
thereby reducing adsorption of geranylgeranylacetone to the wall of the ophthalmic container.
